(19) United States
(12) Patent Application Publication
(10) Pub. No.: US 2012/0041242 A1
(43) Pub. Date: Feb. 16, 2012

(54) SUBSTITUTE NATURAL GAS GENERATION

(76) Inventors: Gregory Joseph Panuccio, East Amherst, NY (US); Vasilis Papavassiliou, Williamsville, NY (US); Raymond Francis Drnevich, Clarence Center, NY (US)

(21) Appl. No.: 12/855,408

(22) Filed: Aug. 12, 2010

(51) Int. Cl. C07C 5/02; B01J 19/00

(52) U.S. Cl. 585/251; 422/149

(57) ABSTRACT

A method and apparatus for producing a substitute natural gas stream to be added to a natural gas stream in which at least part of a refinery gas stream is reacted in a catalytic reactor to hydrogenate olefins into saturated hydrocarbons. The extent to which hydrogen and olefins are reacted in the catalytic reactor is controlled to reduce the concentration of olefins in the resulting substitute natural gas stream and to increase the interchangeability of the substitute natural gas stream with the natural gas. This control can be effectuated by by-passing part of the refinery off gas to an intermediate product stream produced by the catalytic reactor and thereby forming the substitute natural gas stream from the by-passed refinery gas stream and the intermediate product stream.

### FIELD OF THE INVENTION

The present invention relates to a method and system for generating substitute natural gas from a refinery gas. More particularly, the present invention relates to such a method and system in which part of the refinery gas that is used in generating the synthetic natural gas is catalytically reacted to hydrogenate the olefins to saturated hydrocarbons and is combined with a remaining part of the refinery gas by-passing the catalytic reaction to form the resulting substitute natural gas.

### BACKGROUND OF THE INVENTION

Refinery gases are a high quality fuel that is a byproduct of refinery operations. Such off-gases typically contain significant quantities of hydrogen, methane, paraffins, olefins, and nitrogen. Refinery gases may also contain small amounts of carbon monoxide, carbon dioxide and hydrocarbons having five or six carbon atoms and other impurities, for example, heavy metals, chlorides, silica, and sulfur-containing compounds. Refiners typically utilize such gases as a fuel for fired process heaters and boilers. Additionally, if economical, refiners will also recover high value chemicals such as hydrogen and olefins from such off-gases.

Some refineries are fuel long. This means that they have more refinery gases than they can utilize within the refinery without further processing the off-gases. If the refinery is fuel long, then the excess refinery gases will likely either have to be flared or fired in a boiler where the excess steam produced in the boiler is vented. Obviously, the fuel value of the refinery gases when put to such uses or flared will be wasted. A potential value proposition would be to export the refinery gas to a utility for use as a substitute natural gas. However, the refinery gas could not be directly used in such a manner given that utilities have certain specifications with respect to the fuel gas that is delivered to a customer. For example, when the fuel gas is burned, the resultant flame has to be stable with no flashback or lifting and the propensity for incomplete combustion of the fuel as evidence by an increase in carbon monoxide production or the flame having yellow tips. Also, the heat delivered by the flame is also a consideration. These specifications are typically contained in limits of Weaver Indices, AGA Bulletin #36 Indices, or the Wobbe Index of the fuel being produced to customers by the utility. Typically, refinery gases alone do not meet such specifications and thus, can only be combined with natural gas in limited amounts.

Specifically, refinery gases typically contain elevated concentrations of hydrocarbons with more than two carbon atoms which increases both the Wobbe Index and the propensity for incomplete combustion. It can be shown from calculation of Weaver Indices that olefins have a slightly higher propensity for soot and yellow tip formation than their corresponding alkanes, for example, ethylene has a slightly higher tendency to produce yellow tips than ethane. Elevated concentrations of hydrocarbons with two or more carbon atoms also increases the burner flame speed which increases the tendency of the flame to flashback in the burner. It is to be noted that Weaver Index calculations also show that olefins have a slightly higher propensity for flashback than their corresponding alkanes, for example, ethylene has a slightly higher flashback tendency than ethane. Refinery gases will also typically contain elevated concentrations of hydrogen. The presence of hydrogen increases the flame speed and the tendency of the flame to flashback in the burner. Thus, refinery gases can be sold to utilities only to a limited extent because their interchangeability with natural gas is very limited.

As will be discussed, the present invention provides a method and system for treating the refinery gases to increase their interchangeability with natural gas so that they can be sold to a utility to a greater extent than that possible in the prior art.

### SUMMARY OF THE INVENTION

In one aspect, the present invention provides a method of producing a substitute natural gas stream to be added to a natural gas stream. In accordance, with such method, a reactor feed stream is fed to a catalytic reactor. Hydrogen and the olefins within the reactor feed stream are catalytically reacted in the catalytic reactor to produce an intermediate product stream containing saturated hydrocarbons formed from hydrogenation of the olefins. A reactant stream is formed, at least in part, from at least part of a refinery gas stream containing the olefins and at least part of the hydrogen that are reacted in the catalytic reactor. A reactor feed stream is formed, at least in part, from the reactant stream and the reactant stream is heated, prior to forming the reactor feed stream, such that the reactor feed stream has an inlet temperature within a range of between 120° C. and 427° C. upon introduction into the catalytic reactor and the intermediate product stream has a discharge temperature upon discharge from the catalytic reactor within a range of between 204° C. and 650° C. The substitute natural gas stream is formed, at least in part, from at least part of the intermediate product stream. The extent to which the hydrogen and olefins contained in the refinery gas stream are reacted in the catalytic reactor is controlled such that the substitute natural gas stream has a concentration of the olefins that is lower than that of the refinery gas stream and a greater interchangeability with the natural gas than the refinery gas.

The extent to which the hydrogen and olefins contained in the refinery gas stream are reacted in the catalytic reactor can be controlled in part by forming a process stream, at least in part from the refinery gas stream. The reactant stream can be formed, at least in part, from part of the process stream. A by-pass stream can be formed, at least in part, from a remaining part of the process stream. The by-pass stream is combined with the at least part of the intermediate product stream after having been cooled, thereby to form a mixed gas stream and the substitute natural gas stream is formed from at least part of the mixed gas stream. The flow rate of the by-pass stream is controlled such that as the flow rate is increased, less of the olefins are reacted within the catalytic reactor and when the flow rate is decreased more of the olefins are reacted within the catalytic reactor.

The process stream can be composed solely of the refinery gas stream, the reactant stream can be composed solely of the part of the process stream, the reactor feed stream can be composed solely of the reactant stream, the by-pass stream can be composed solely of the remaining part of the process stream the by-pass stream can be combined with all of the intermediate product stream and the substitute natural gas stream can be formed from all of the mixed gas stream. Alternatively, a supplemental process stream is combined with the refinery gas stream to form the process stream. In another alternative, the reactant stream can be formed by combining the part of the process stream with part of a supplemental process stream and the by-pass stream is formed by combining the remaining part of the process stream with the remaining part of the supplemental process stream. In the foregoing alternatives, the supplemental process stream is nitrogen, air, hydrogen, natural gas or combinations thereof.

The reactant stream can be combined with steam after having been heated to form the reactor feed stream. Additionally, the by-pass stream can be combined with the part of intermediate product stream and the substitute natural gas stream can be formed from the mixed gas stream. In such case, a recycle stream is formed from a remaining part of the intermediate product stream either before all of the intermediate product stream is cooled or the part of the intermediate product stream is cooled and the recycle stream is introduced into the catalytic reactor. In yet another alternative, the by-pass stream can be combined with all of the intermediate product stream after having been cooled. In such case, the substitute natural gas stream is formed from the part of the mixed gas stream and a recycle stream is formed from a remaining part of the mixed gas stream. In either of the foregoing two alternatives, steam can also be combined with the reactant stream to form the reactor feed stream.

The substitute natural gas stream can be formed by combining at least part of the intermediate product stream with a supplemental process stream after the intermediate product stream is cooled and the supplemental process stream is nitrogen, air, hydrogen, natural gas or combinations thereof. One of the reactant stream and the reactor feed stream is also formed from a supplemental process stream and the supplemental process stream is nitrogen, air, hydrogen, natural gas, steam or combinations thereof.

In another aspect, the present invention provides an apparatus for producing a substitute natural gas stream to be added to a natural gas stream. A catalytic reactor, configured to catalytically react hydrogen and the olefins within a reactor feed stream, is provided to produce an intermediate product stream containing saturated hydrocarbons formed from hydrogenation of the olefins. A flow network connected to the catalytic reactor and has an inlet to receive the refinery gas stream, an outlet to discharge the substitute natural gas stream, a preheater positioned between the inlet and the catalytic reactor and an after-cooler positioned between the catalytic reactor and the outlet. The flow network is configured such that a reactant stream, formed at least in part from at least part of the refinery gas stream, is introduced into the preheater, the reactor feed stream is formed, at least in part, from the reactant stream after having been heated and the substitute natural gas stream is formed at least in part from at least part of the intermediate product stream after having been cooled in the after-cooler. The preheater is configured to heat the reactant stream such that the reactor feed stream has an inlet temperature within a range of between 120° C. and 427° C. upon introduction into the catalytic reactor and the intermediate product stream has a discharge temperature upon discharge from the catalytic reactor within a range of between 204° C. and 650° C. A means is provided for controlling an extent to which the hydrogen and olefins contained in the refinery gas stream are reacted in the catalytic reactor such that the substitute natural gas stream has a concentration of the olefins that is lower than that of the refinery gas stream and a greater interchangeability with the natural gas than the refinery gas.

The controlling means at least in part can comprise a by-pass line having one end positioned within the flow network between the inlet and the preheater and the other end positioned between the after-cooler and the outlet. A flow control valve is located within the by-pass line such that when the flow control valve is set in an open position the reactant stream is composed of part of a process stream that is composed at least in part of the refinery gas stream, a by-pass stream is formed from at least a remaining part of the process stream flows within the by-pass line and combines with the at least part of the intermediate product stream to form a mixed gas stream and the substitute natural gas stream is formed, at least in part, from at least part of the mixed gas stream. The flow control valve is configured to be progressively opened from a closed position such that flow of the by-pass stream increases and the hydrogen and the olefins are progressively reacted within the catalytic reactor to a lesser extent.

The flow network can be configured such that the process stream is composed solely of the refinery gas stream, the reactant stream is composed of the part of the process stream, the reactor feed stream is composed solely of the reactant stream, the by-pass stream is composed solely of the remaining part of the process stream, the by-pass stream is combined with all of the intermediate product stream, and the substitute natural gas stream can be formed from all of the mixed gas stream. In an alternative, the flow network has a supplemental inlet that is positioned between the inlet and the preheater to receive a supplemental process stream that combines with the refinery gas stream to form the process stream. In another alternative, the flow network has a supplemental inlet to receive a supplemental process stream, a first flow path and a second flow path. The first flow path communicates, at one end with the supplemental inlet and at the other end to a position within the flow network between the inlet and the preheater such that the part of the process stream combines with part of the supplemental process stream to form the reactant stream. The second flow path communicates, at one end, with the supplemental inlet and at the other end with the by-pass line, downstream of the flow control valve, such that the remaining part of the process stream combines with the remaining part of the supplemental process stream to form the by-pass stream. The supplemental process stream is nitrogen, air, hydrogen, natural gas or combinations thereof.

Additionally, the flow network can have a supplemental inlet to receive steam. This supplemental inlet is positioned between the inlet and the catalytic reactor such that the reactant stream combines with the steam after having been heated to form the reactor feed stream.

A recycle line can be positioned, at one end, between the catalytic reactor and the other end of the by-pass line. At the other end, the recycle line can be positioned between the one end of the by-pass line and the catalytic reactor. The recycle line has a blower such that the remaining part of the intermediate product stream flows within the recycle line as a recycle stream back to the catalytic reactor. In such case the mixed gas stream is formed from the by-pass stream and the part of the intermediate product stream and the substitute natural gas stream is formed from the mixed gas stream. Alternatively, the other end of the by-pass line can be positioned between the outlet and the after-cooler such that the by-pass stream combines with all of the intermediate product stream after having been cooled. In such case, the recycle line is positioned, at one end, between the by-pass line and the outlet such that the substitute natural gas stream is formed from the part of the mixed gas stream and a remaining part of the mixed gas stream flows into the recycle line as a recycle stream. The other end of the recycle line is positioned between the one end of the by-pass line and the catalytic reactor such that the recycle stream flows back to the catalytic reactor under impetus of the blower. In both embodiments concerning the recycle stream, a supplemental inlet can be provided to receive steam, the supplemental inlet in communication with the recycle line such that the steam is also introduced into the catalytic reactor along with the recycle stream.

### BRIEF DESCRIPTION OF THE DRAWINGS

While the specification concludes with claims distinctly pointing out the subject matter that Applicants regard as their invention, it is believed that the invention will be better understood when taken in connection with the accompanying drawings in which:

FIG. 1 is a schematic illustration of an apparatus for carrying out a method in accordance with the present invention;

FIG. 2 is a schematic illustration of an alternative embodiment of an apparatus for carrying out a method in accordance with the present invention;

FIG. 3 is a schematic illustration of an alternative embodiment of FIG. 2; and

FIG. 4 is a schematic illustration of an alternative embodiment of FIG. 3.

### DETAILED DESCRIPTION

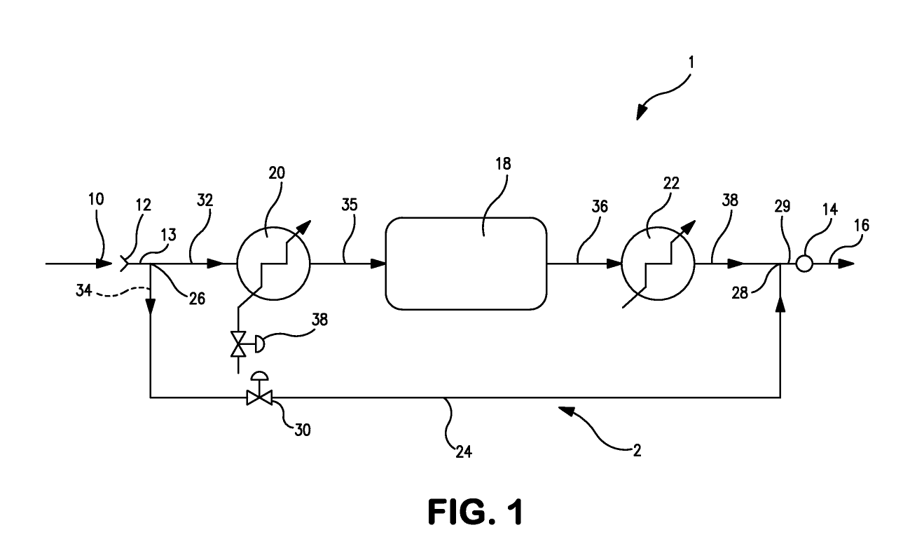

With reference to FIG. 1, an apparatus 1 is disclosed for catalytically reacting part of the olefins and hydrogen that are contained within a refinery gas stream 10 to produce a substitute natural gas stream 16 that is more interchangeable with natural gas than the refinery gas stream due to the consumption of hydrogen and olefins in the catalytic reaction.

Typical refinery gas streams 10 can be a fluidic catalytic cracker (“FCC”) off-gas, a sweet refinery gas (“SRG”), coker off-gas or other type of off-gas containing high amounts of hydrocarbons with more than two carbon atoms. It can also be a combination of more than one stream that has been treated in an amine system to reduce sulfur species to low levels and it is then typically referred as sweet refinery gas. All of such off-gases contain a significant quantity of olefins and hydrogen that would restrict use of such streams in a utility application in which the fuel gas utilized by such a utility must meet specific combustion specifications.

In accordance with the present invention, apparatus 1 is provided with a flow network 2 having an inlet 12 to receive refinery gas stream 10 and an outlet 14 to discharge the substitute natural gas stream 16. Connected within the flow network 2 is a catalytic reactor 18, a preheater 20 positioned between the catalytic reactor 18 and the inlet 12 and an after-cooler 22 positioned between the catalytic reactor 18 and the outlet 14. The flow network 2 also has a by-pass line 24. One end 26 of the by-pass line 24 is positioned within the flow network 2 between the inlet 12 and the preheater 20 and the other end 28 positioned between the after-cooler 22 and the outlet 14. A flow control valve 30 is located within the by-pass line 24.

In flow network 2, the refinery gas stream 10 is introduced into inlet 12 to produce a process stream 13 that is divided into a reactant stream 32 that is introduced into preheater 20 by way of a conduit or other connection between the preheater 20 and the inlet 12 and a by-pass stream illustrated by arrowhead 34 that flows within the by-pass line 24. Preheater 20 preheats the reactant stream 32 to produce a reactor feed stream 35 that flows in a conduit of the flow network 2 or other connection between the catalytic reactor 18 and the preheater 20. The catalytic reactor 18 is configured to catalytically react the hydrogen and the olefins within the reactor feed stream 35 through hydrogenation to produce a intermediate product stream 36 containing resulting saturated hydrocarbons that flows within a conduit or other connection between catalytic reactor 18 and the after-cooler 22. The intermediate product stream 36 after having been cooled in the after-cooler 22 joins up with the by-pass stream 34, as a cooled intermediate product stream 38, by means of the by-pass line 24 to produce a mixed gas stream 29 that is discharged from the outlet 14 as the substitute natural gas stream 16.

The flow control valve 30 controls the flow rate of the by-pass stream 34 flowing within the by-pass line 24 and also, the flow rate of the reactant stream 32 and the reactor feed stream 35 introduced into catalytic reactor 18. Obviously, the greater the flow rate of the by-pass stream 34, the lower the flow rate of the reactor feed stream 35 and vice-versa. As the flow rate of the by-pass stream 34 is decreased, the quantity of the olefins and hydrogen available for reacting within the catalytic reactor is progressively increased and the substitute natural gas stream 16 will have a greater content of saturated hydrocarbons and lower content of olefins than the refinery gas stream 10. As will be discussed, this will increase the interchangeability of the substitute natural gas stream 16 with a natural gas stream to be supplied to a utility. However, this would also affect the heating value of the substitute natural gas stream 16. For example, if all of the hydrogen were reacted, then on a unit basis, that is heating value per cubic feet, the heating value would increase. This increase in heating value might in fact be large enough to make the substitute natural gas stream 16 less interchangeable with the natural gas. However, since the reaction would decrease the volume of the substitute natural gas stream 16 being supplied to a utility on an hourly basis, then on such hourly basis the heating value would actually decrease. The problem with such a decrease is that a substitute natural gas is sold to the utility on the heating value when viewed on an hourly basis. Consequently, by controlling the flow of the by-pass stream 34, interchangeability will be increased through hydrogenation of the olefins to the saturated hydrocarbons, yet the substitute natural gas stream will also be formed in part through a portion of the refinery gas stream 10 that has not been reacted so that the heating value on an hourly basis of substitute natural gas stream 16 is maximized while the interchangeability of substitute natural gas stream 16 meets utility specifications.

It is possible, however, to close flow control valve 30 completely. In such case all of the refinery gas stream 10 will enter the catalytic reactor 18. In fact, embodiments of the present invention are possible without the by-pass stream 24. In such case, the amount of preheating supplied by preheater 20 would then be the sole control of the degree to which hydrogen and olefins react within catalytic reactor 18. Typically, preheater 20 will be a steam driven preheater in which the reactant feed is preheated by superheated steam. Controlling the flow rate of the steam would control the amount of preheating delivered by preheater 20. A control valve 38 can be provided for such purposes. The same would hold true if other high temperature streams available at a refinery site were used for such purposes. However, this control would have to at least supply heat to heat the reactor feed stream 35 to a temperature that will ensure that the catalyst within the catalytic reactor 18 will promote the hydrogenation reactions. This temperature has an upper limit, namely, the temperature at which the catalyst would deactivate due to the sintering of the catalyst, typically 650° C. For such purposes, the inlet temperature will be controlled in a typical range of between about 120° C. and about 427° C. This range will ensure that the intermediate product stream 30 is at a temperature in a range of between about 204° C. and about 650° C. This being said, embodiments of the present invention are also possible without such preheating control and the sole handle of control could be supplied by flow control valve 30.

Catalytic reactor 18 contains a partial oxidation catalyst which is preferably a metallic monolith coated with a catalytic layer that contains platinum, rhodium, palladium, nickel or ruthenium. The structure of the monolith can be reticulated foam, honeycomb or a corrugated foil wound in a spiral configuration. Catalyst coated beads or ceramic monoliths in the form of a reticulated foam or honeycomb structure are other possibilities. It is believed that the metallic supported catalyst has better performance than other supported catalyst in that it has better heat conductivity, a more uniform temperature profile than other catalyst forms and a lower operating temperature. All of these factors permit the more selective conversion of olefins without converting too much of the paraffins, for instance, ethane, into olefins. A useful catalyst can be obtained from Sud-Chemie of Louisville, Ky., United States of America, which is in the form of a monolith which is sold as PC-PDX 1 on FeCrAlY.

The catalytic reactor 18 should be sized such that the total gas hourly space velocity calculated at 60° F. and 14.696 psia is between 25,000 and 200,000 hr⁻, most preferably 50,000 hr⁻¹. In this regard, for such purposes, space velocity is defined as the ratio of the volumetric gas flow rate at standard temperature and pressure divided by the empty reactor volume.

The term, “interchangeable” as used herein and in the claims means the degree to which a natural gas stream can be replaced by a substitute natural gas stream; and the degree to which the substitute natural gas stream is interchangeable with natural gas, concerns the specification used by the utility for a fuel gas to be supplied to customers. One common utility specification is given by the Weaver Indices, Jₓ. There are six of such indices and consequently, x is: “H” the heating value; “A” the air flow required to combust the stream; “L” the lifting index which is a measure where the base of a flame comes off a burner; “I” a measure of incomplete combustion; “F” flame flashback; and “Y” the amount of yellowing in the tip of the flame.

Another common utility specification for determining substitute natural gas interchangeability with natural gas is given by the AGA Bulletin #36 Indices. Similar to the Weaver Indices, the Bulletin #36 Indices can be used to determine interchangeability based on flame lifting (I_{L}), flame flashback (I_{F}), and yellow tip formation (I_{F}). The Wobbe Index can also be used to determine substitute natural gas interchangeability; however the Wobbe Index only accounts for deviation in the heating value and specific gravity of the substitute natural gas and does not incorporate differences due to flashback, lifting, or yellow-tipping. The Weaver Indices will be used herein to demonstrate the utility of the current invention with respect to substitute natural gas interchangeability.

In the utility specification there are ideal values and there exists a tolerance by which a fuel stream can deviate from the ideal values. When the Weaver Indices of the substitute natural gas stream are compared with those of a particular natural gas stream, the greatest deviation in the Weaver indices will set the interchangeability of the substitute natural gas with the natural gas and therefore the flow rates at which the substitute natural gas can be combined with the natural gas. An equation for this can be expressed as follows:

Interchangeability_{X}=100·(J_{X,R}−J_{X,NG})/(J_{X,S}−J_{X,NG})

Where Interchangeability_{X} is the percentage of total natural gas plus substitute natural gas mixture that can be composed of the substitute natural gas.
In this equation, “R” is the recommended value for a particular Weaver Index or in other words the tolerance, “S” is the calculated value for the substitute natural gas and “NG” is the value of the particular Weaver Index for the particular natural gas to be augmented by the substitute natural gas.

By way of example, Table 1 gives a stream summary for the reactor system 1. In this example, the refinery gas stream 10 is split such that the by-pass stream 34 has a flow rate of 460 mscfh (thousand standard cubic feet per hour at 60° F. and 14.696 psia) and the reactant stream 32 has a flow rate of 540 mscfh. The reactant stream 32 is heated in preheater 20 such that the reactor feed stream 35 has a temperature of 204° C. The hydrogen and olefins are catalytically reacted within catalytic reactor 18 to form alkanes and release heat per the exothermic hydrogenation reaction. The resulting intermediate product stream has a temperature of 528° C. while the hydrogen concentration decreased from 30 percent to 18 percent and the total olefin concentration decreased from 15 percent to 0.4 percent. The intermediate product stream 36 is then cooled in after-cooler 22 and combines as stream 38 with the by-pass stream 34 to form the substitute natural gas stream 16.

TABLE 1

| Stream | 10 | 34 | 32 | 35 | 36 | 38* | 16 |
|---|---|---|---|---|---|---|---|
| Temperature ° C. | 38 | 38 | 38 | 204 | 528 | 38 | 38 |
| Flow, mscfh | 1000 | 460 | 540 | 540 | 461 | 461 | 921 |
| HHV, btu/scf | 1108 | 1108 | 1108 | 1108 | 1272 | 1272 | 1190 |
| Composition, mol % | | | | | | | |
| H₂ | 30.0 | 30.0 | 30.0 | 30.0 | 18.0 | 18.0 | 24.0 |
| N₂ | 5.0 | 5.0 | 5.0 | 5.0 | 5.9 | 5.9 | 5.4 |
| CH₄ | 30.0 | 30.0 | 30.0 | 30.0 | 35.2 | 35.2 | 32.6 |
| C₂H₆ | 13.0 | 13.0 | 13.0 | 13.0 | 26.9 | 26.9 | 20.0 |
| C₃H₈ | 5.0 | 5.0 | 5.0 | 5.0 | 10.3 | 10.3 | 7.7 |
| n C₄H₁₀ | 2.0 | 2.0 | 2.0 | 2.0 | 3.4 | 3.4 | 2.7 |
| C₂H₄ | 10.0 | 10.0 | 10.0 | 10.0 | 0.1 | 0.1 | 5.0 |
| C₃H₆ | 4.0 | 4.0 | 4.0 | 4.0 | 0.2 | 0.2 | 2.1 |
| n C₄H₈ | 1.0 | 1.0 | 1.0 | 1.0 | 0.1 | 0.1 | 0.5 |

*Note:
Intermediate product stream after having passed through the after cooler 32

Table 2, below, shows calculated values for Weaver Index (J_{X}) and Interchangeability for refinery gas stream 10 and substitute natural gas stream 16 in the current example. In this example, it was assumed that the Natural Gas, “NG” has ideal values of the Weaver indices.

TABLE 2

| Weaver | J_{X, S} | | Interchangeability_{X} | | Weaver Index Values | |
|---|---|---|---|---|---|---|
| Index | Stream 10 | Stream 16 | Stream 10 | Stream 16 | NG | Recommended |
| J_{H} | 1.02 | 1.05 | 100 | 100 | J_{H} = 1 | 0.95 < J_{H} < 1.05 |
| J_{A} | 0.98 | 1.02 | 100 | 100 | J_{A} = 1 | 0.8 < J_{A} < 1.2 |
| J_{L} | 1.83 | 1.65 | 100 | 100 | J_{L} = 1 | J_{L} > 0.64 |
| J_{F} | 0.88 | 0.56 | 30 | 46 | J_{F} = 0 | J_{F} < 0.26 |
| J_{Y} | 0.54 | 0.55 | 55 | 54 | J_{Y} = 0 | J_{Y} < 0.30 |
| J_{I} | 0.03 | 0.07 | 100 | 70 | J_{I} = 0 | J_{I} ≦ 0.05 |

As is apparent from Table 2, the tendency for flame flashback (J_{F}) limits the interchangeability for both refinery gas stream 10 and substitute natural gas stream 16 to thirty percent and 46 percent, respectively. As such, for every 100 cubic feet per hour of a fuel stream to be used by a utility, roughly, 46 percent could be made up of the substitute natural gas stream 16, remainder natural gas, versus 30 percent if just the refinery gas stream 10 were exported to the utility. Also, another important feature of the present invention that is inherent in Table 2 concerns the by-pass of part of the refinery gas stream 10. If all of the refinery gas stream 10 were catalytically reacted, then the resulting substitute natural gas would contain little if any hydrogen and long chain saturated hydrocarbons containing two or more carbon atoms. The problem with such a stream is that the long chain hydrocarbons when combusted would tend to form soot and thus, the yellowing factor “J_{Y}” would be limiting. The lack of hydrogen would increase the heating value of such stream as measured by “J_{H}” to too high a level. Consequently by removing substantially all of the hydrogen and olefins through catalytic hydrogenation reactions, the resulting stream would be less interchangeable than the resulting substitute natural gas stream 16 produced by the present invention.

Table 3 below gives a summary of the economic benefit of processing a refinery gas according to the current invention in accordance with the Example, discussed above. The “Baseline” scenario summarizes the economics for the case where refinery gas is potentially exported to the utility without further processing. The low interchangeability of the export gas in the “Baseline” scenario limits the RFG export to 656 mmbtu/hr. The remaining 451 mmbtu/hr of RFG must be flared or, if flaring is not an option due to environmental constraints, then refinery operations would have to be curtailed. If exported RFG can be sold to the utility for $4.5/mmbtu, then the income from RFG sales for the ‘Baseline’ case is $2954/hr. In the Example, the substitute natural gas allows 1096 mmbtu/hr of refinery gases to be sold to the utility and no flaring. Approximately 12 mmbtu/hr of heat is transferred from the RFG to the heat recovery system in shown ‘Example 1’. If the RFG is sold to the utility at $4.5/mmbtu, then the income from RFG sales for the “Example” scenario is $4931/hr. The incremental income that results from the additional RFG sales for “Example 1” relative to the “Baseline” scenario is $1978/hr. RFG sales increase by 67 percent when the gas is processed according to the current invention.

TABLE 3

| Scenario | Baseline | Example |
|---|---|---|
| Total Utility Pipeline Demand, mscfh | 2000 | 2000 |
| RFC Available for Export, mscfh | 1000 | 1000 |
| RFC Available for Export, mmbtu/hr | 1108 | 1108 |
| RFC Export Interchangeability, % | 30 | 46 |
| RFC Exported to Utility, mmbtu/hr | 656 | 1096 |
| RFC to Flare, mmbtu/hr | 451 | 0 |
| Income from RFC Sales, $/hr | 2954 | 4931 |
| Additional RFC Sales Income, $/hr | — | 1978 |

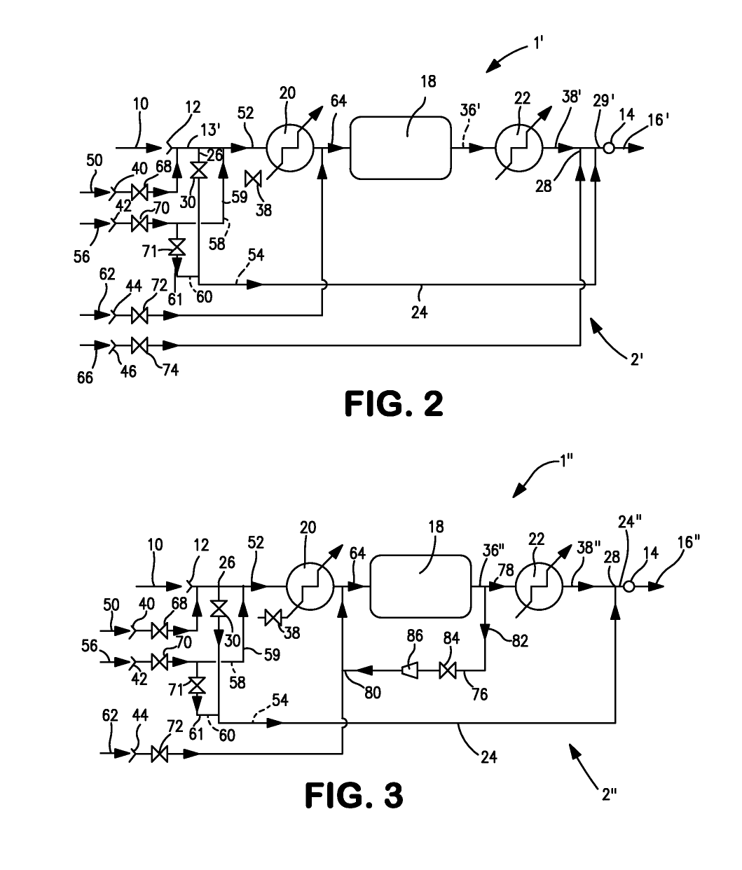

With reference to FIG. 2, an apparatus 1′ is illustrated having some of the same components as apparatus 1, namely, the inlet 12, the outlet 14, the preheater 20 and related control valve 38, the catalytic reactor 18, the after-cooler 22 and the by-pass line 24 and related control valve 30. All of these components have been discussed above and have the same function and configuration in apparatus 2. The flow network 2′ provided for apparatus 1′ is modified with the addition of supplemental inlets 40, 42, 44 and 46 and related control valves for purposes that will be discussed hereinafter.

Supplemental inlet 40 is provided to receive a supplemental process stream 50 that can be nitrogen, air, hydrogen, natural gas or a combination of such streams. As illustrated supplemental inlet 40 is positioned within the flow network 2′ between the inlet 12 and the preheater 20 and preferably, before end 26 of the by-pass line 24 such that supplemental process stream 50 will combine with the refinery gas stream 10 to produce a process stream 13′. As a result, reactant stream 52 can be made up of both the supplemental process stream 50 and the refinery gas stream 10 and the by-pass stream 54 can be formed, at least in part, from the supplemental process stream 50 as well as a part 60 of a further supplemental process stream 56. The addition of nitrogen has the advantage of acting as a diluent to control temperatures within catalytic reactor 18 and also to reduce the flame speed, sooting and incomplete combustion tendencies of the substitute natural gas stream 16′ discharged from outlet 14. Air will act as a diluent to reduce the Wobbe index, flame speed, sooting and incomplete combustion tendencies of the resulting substitute natural gas stream 16′. Hydrogen addition will have the effect of converting more of the olefins to saturated hydrocarbons. Additionally, even if there is sufficient hydrogen to convert all of the olefins within the refinery gas stream 10, an increase in the hydrogen content within the substitute natural gas stream 16′ will have the effect of reducing the Wobbe index and the tendency for incomplete combustion and soot formation through dilution of resulting saturated hydrocarbons having two or more carbon atoms. Natural gas addition will dilute the concentration of olefins and hydrogen within the reactant stream 52 to in turn reduce the maximum temperature within catalytic reactor 18. The interchangeability of the substitute natural gas should be calculated excluding the diluent natural gas used within catalytic reactor 18 since the natural gas will likely be obtained from the same utility to which the substitute natural gas will be sold.

A supplemental process stream 56 can be introduced into inlet 42. Supplemental process stream 56 can also be nitrogen, air, hydrogen, natural gas or a combination of such streams and can be used in place of or in addition to process stream 50. A portion 58 of supplemental process stream 56 can be combined with the remaining part of the process stream 13′ so as to form part of the reactant stream 52. A portion 60 of supplemental process stream 56 can be used to form part of the by-pass stream 54. As illustrated flow network 2′ is provided with flow paths 59 and 61 for flow of the portions 58 and 60 of the supplemental process stream 56. Supplemental process stream 56 can provide benefits that are similar to supplemental process stream 50, with certain advantages. These advantages arise from the ability to control the division of such stream between the catalytic reactor 18 and the by-pass stream 54 independently of the flow of stream 13 to the catalytic reactor 18 and the by-pass stream 54. For example, if supplemental process stream 56 is made up of hydrogen, portion 58 can act to increase conversion of olefins to saturated hydrocarbons within catalytic reactor 18 while portion 60 can act to reduce the tendencies for incomplete combustion, sooting and a Wobbe index that is too low in the substitute natural gas stream 16′.

A supplemental process stream 62 can be introduced into supplemental inlet 44 and can be made up of steam. The steam acts as a diluent to reduce the maximum temperature within catalytic reactor 18. The steam can also suppress coke formation and scrub coke from the catalyst surface by gasifying adsorbed carbonaceous deposits. For such purposes, supplemental inlet 44 is positioned between the inlet 12 and the catalytic reactor and preferably, as illustrated, between the preheater 20 and the catalytic reactor 18 such that the reactor feed stream 64 can be made up, in part, of the supplemental process stream 62 or steam.

Additionally, a supplemental process stream 66 can be introduced into the cooled intermediate product stream 38′ to form a mixed gas stream 29′ that will be discharged from the outlet 14 as the substitute natural gas stream 16′. Supplemental process stream 66 can be nitrogen, air, hydrogen, natural gas or a combination of such streams. For such purposes, the supplemental inlet 46 is positioned within the flow network 2′ between end 28 of the by-pass line 24 and the outlet 14.

Any and all of the supplemental process streams 50, 56, 62 and 66 are optional. If present control of such streams is provided by their respective control valves 68, 70, 71, 72 and 74 that can be operated to control flow and also have a shut-off position in which the flow of each and any of such streams will be shut-off. If all of such control valves are set in the shut-off position, then an operation is obtained that is the same as that described above with respect to apparatus 1 in FIG. 1. If control valves 68 and 70 are set in the closed position, then reactant stream 52 will be made up of solely the portion of the refinery gas stream 10 that is not used in forming the by-pass stream 54. If, however, control valve 70 were then opened, then the reactant stream 52 would be formed in part from the supplemental process stream 56 and, if control valve 71 were opened, then the by-pass stream would also be formed in part of the supplemental process stream 56. As illustrated control valve 71 is positioned within flow path 61 and controls the flow of portions 58 and 60 of the supplemental process stream 56 as such control valve is progressively opened. If control valve 68 were opened, then the by-pass stream 24 and the reactant stream 52 would made up in part from portions of both supplemental process stream 50 and the supplemental process stream 56. While as indicated above, both of such supplemental process streams 50 and 56 could be selected from the same group of components, the inclusion of both inlets 40 and 42 allow for greater flexibility in the connection of apparatus 1′ within the refinery, for example, inlet 40 could be provided with one component and inlet 42 could be provided with another component. The opening of control valve 72 would introduce the supplemental process stream 62 into the reactant stream 52 after having been heated in preheater 20 and thereby form part of the reactor feed stream 64. When control valve 74 is set in an open position, then part of the substitute gas stream 16′ is also formed in part from the supplemental gas stream 66.

Referring to FIG. 3, an apparatus 1″ is illustrated that incorporates a flow network 2″ that has the features of flow network 2′ shown in FIG. 2 with the addition of a recycle line 76 and without the supplemental inlet 46 and associated control valve 74, although the same could be included. Recycle line 76 is positioned within flow network 2″, at one end 78 between the catalytic reactor 18 and the other end 28 of the by-pass line 24. As illustrated, the one end 78 is located between the catalytic reactor 18 and the after-cooler 22. However, the one end 78 could be positioned after the after-cooler 22. The other end 80 of the by-pass line 24 is positioned within flow network 2″ between the one end 26 of the by-pass line and the catalytic reactor 18 such that a recycle stream 82 flowing within the recycle line 76 is introduced into the catalytic reactor. Preferably, as illustrated, the other end 80 of the recycle line is connected within flow network 2″ such that the recycle stream 82 joins up with supplemental process stream 62 if present and in any case forms part of the reactor feed stream 64 to be fed into catalytic reactor 18. However, such other end 80 of the recycle line 76 could be positioned between the one end 26 of the recycle line 24 and the preheater 20 so that part of the reactant stream 52 is additionally formed from the recycle stream 82.

Recycle stream 82 is made up of part of an intermediate product stream 36″ to act as a diluent within the catalytic reactor 18 since recycle stream 82 will contain a lower concentration of olefins than the part of the refinery gas stream 10 that is being fed into the catalytic reactor 18. Such diluent can be added to control temperatures within catalytic reactor 18. Thus, in such embodiment, a mixed gas stream 29″ that is discharged from outlet 14 as the substitute natural gas stream 16″ is formed from the by-pass stream 54 and part of the cooled intermediate product stream 38″. Flow of the recycle stream 82 can be controlled by a control valve 84 and recirculated with the aid of a blower 86 or if necessary, a compressor. In the illustrated embodiment, the recycle stream 82 is then combined with the reactant stream 52 and optionally supplemental process stream 44 which is made up of steam. Control valves 72 and 84 can be control valves and can be used to selectively allow the combination of recycle stream 82 and supplemental process stream 44, the addition of recycle stream 82 alone to the catalytic reactor 18 or the addition of the supplemental process stream 44 alone to the reactant stream 52 or varying combinations of the two streams that might be required based on changes in the composition of stream 10.

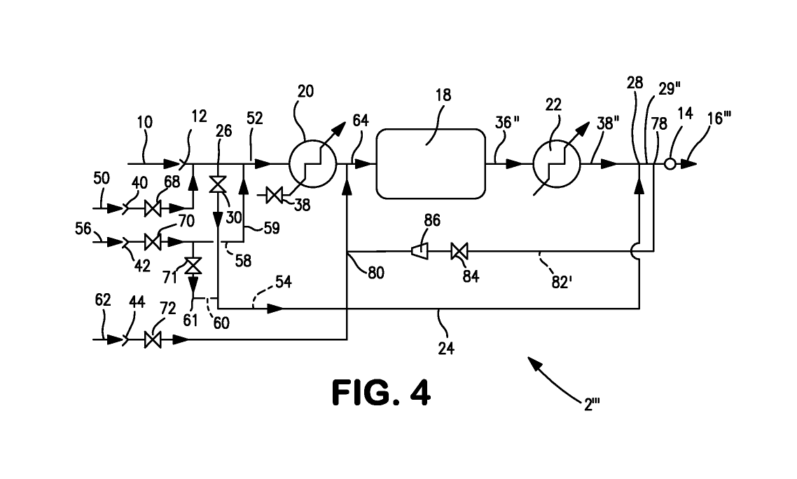

With additional reference to FIG. 4, an alternative embodiment of FIG. 3 is shown in which the one end 78 of the recycle line 82 is positioned within a flow network 2′″ between the other end 28 of the by-pass line 24 and the outlet 14. In such embodiment the substitute natural gas stream 16′″ is formed from part of the mixed gas stream 29″ and a remaining part of the mixed gas stream 29″ thereby forms a recycle stream 82′. Recycle stream 82′ will thereby act as a diluent that has a make up that is provided by both the cooled intermediate product stream 38′″ and the by-pass stream 54.

With reference again to FIG. 1, the ratio of flow rates between the reactant stream 32 and the by-pass stream 34 should be optimized subject to the composition of the refinery gas stream 10, the total available flow of the refinery gas stream 10, and the total demand of for the fuel stream to be made up of natural gas and the substitute natural gas stream 16 which in practice would be a natural gas utility pipeline to which the substitute natural gas stream 16 is exported. If possible, the flow of the refinery gas through catalytic reactor 18 should be minimized such that the interchangeability of substitute natural gas stream 16 is just high enough so that all available refinery gas is utilized. Increasing the portion of the refinery gas that goes to make up the reactor feed stream 35 beyond the minimum amount required may increase the interchangeability of the resulting substitute natural gas stream 16 but will decrease the net income as the total heat flow in the substitute natural gas stream 16 will decrease since the reactions in the catalytic reactor are exothermic. As can be appreciated, the same considerations would apply to apparatus 1′ and the split of refinery gas between the by-pass stream 54 and the reactor feed stream 64.

Therefore, the control of the split through appropriate control of control valve 30 for apparatus 1 and apparatus 2 is important not only for carrying out the present invention, but also, to maximize economic benefits from the invention. The control set points for the process should be optimized according to the following procedure. A model is developed which accurately predicts process performance by using data from laboratory experiments or field operations. The model could be built as, for example, a look-up table, an equilibrium limited reactor, or a kinetically limited reactor. The process model can then be used to calculate optimum process control set points with the objective of increasing the interchangeability of substitute natural gas stream 16 or 16′ just enough to maximize the flow of the refinery gas stream 10 without exceeding process constraints while minimizing the cost of operating the process. The inputs of the process model are the desired flow of refinery gas stream 10, the composition of the refinery gas stream 10, and the demand for total utility pipeline gas. The desired flow of refinery gas stream 10 is determined from refinery operations, the composition of the refinery gas stream 10 can be obtained by analyzing a sample of the gas (for example in a gas chromatograph), and the demand for total utility pipeline gas is obtained from the utility to which substitute natural gas stream 16 or 16′ is exported. The total utility pipeline gas demand and desired flow of refinery gas stream 10 to determine the interchangeability required for substitute natural gas stream 16 or 16′ and the composition of refinery gas stream 10 will have an effect on optimum control set points.

With respect to FIG. 2, a greater degree of control is possible and the control set points that will be optimized for apparatus 1′ and the process conducted therein are the flow rates of the refinery gas stream 10 and the flow rates of supplemental process stream 50, 56, 62 and 66 as well as parts 58 and 60 of supplemental process stream 56. All of the flows of such stream are controlled with the use of control valves 68, 70, 71, 72 and 74. It is to be noted, however, that in practice, the flow rates of the refinery gas stream 10 and the supplemental process stream 50, 56, 62 and 66 are constrained by maximum availability. As discussed above, the temperature of reactor feed stream 64 and also 35 are constrained by the minimum temperature required for reaction in catalytic reactor 18 and the maximum attainable temperature given the heating medium used in preheater 20. The temperature of the intermediate product streams 36 and 36′ are constrained by the maximum allowable working temperature of catalytic reactor 16. There will also be constraints on the maximum available heating duty in preheater 20 and the maximum available cooling duty in after-cooler 22 based on the design of the installed equipment. There may be constraints on the properties of substitute natural export gas streams 16 and 16′ such as (but not limited too) minimum and maximum Wobbe Index, minimum and maximum heating value, maximum hydrocarbon dewpoint, maximum flame speed, and maximum concentrations of inert species (e.g. N₂ and CO₂), CO, or O₂.

The composition of substitute natural gas streams 16 and 16′ should be monitored to ensure that the performance predicted by the process model matches actual performance. The parameters in the model will invariably have to be adjusted to reflect changing performance over time.

While the present invention has been described with reference to preferred embodiments, as will occur to those skilled in the art, numerous changes, additions and omissions can be made without departing from the spirit and scope of the invention as set forth in the appended claims.

We claim:

1. A method of producing a substitute natural gas stream to be added to a natural gas stream, said method comprising:
feeding a reactor feed stream to a catalytic reactor and catalytically reacting hydrogen and the olefins within the reactor feed stream to produce an intermediate product stream containing saturated hydrocarbons formed from hydrogenation of the olefins;
forming a reactant stream, at least in part, from at least part of a refinery gas stream containing the olefins and at least part of the hydrogen that are reacted in the catalytic reactor;
forming the reactor feed stream, at least in part, from the reactant stream;
heating the reactant stream, prior to forming the reactor feed stream, such that the reactor feed stream has an inlet temperature within a range of between 120° C. and 427° C. upon introduction into the catalytic reactor and the intermediate product stream has a discharge temperature upon discharge from the catalytic reactor within a range of between 204° C. and 650° C.;
cooling the intermediate product stream and forming the substitute natural gas stream, at least in part, from at least part of the intermediate product stream;
controlling an extent to which the hydrogen and olefins contained in the refinery gas stream are reacted in the catalytic reactor such that the substitute natural gas stream has a concentration of the olefins that is lower than that of the refinery gas stream and a greater interchangeability with the natural gas than the refinery gas.

2. The method of claim 1, wherein the extent to which the hydrogen and olefins contained in the refinery gas stream are reacted in the catalytic reactor is controlled in part by:
forming a process stream, at least in part, from the refinery gas stream;
forming the reactant stream, at least in part, from part of the process stream;
forming a by-pass stream, at least in part, from a remaining part of the process stream;
combining the by-pass stream with the at least part of the intermediate product stream after having been cooled, thereby to form a mixed gas stream;
forming the substitute natural stream from at least part of the mixed gas stream; and
controlling flow rate of the by-pass stream such that as the flow rate is increased, less of the olefins are reacted within the catalytic reactor and when the flow rate is decreased more of the olefins are reacted within the catalytic reactor.

3. The method of claim 2, wherein the process stream is composed solely of the refinery gas stream, the reactant stream is composed solely of part of the process stream, the reactor feed stream is composed solely of the reactant stream, the by-pass stream is composed solely of the remaining part of the process stream, the by-pass stream is combined with all of the intermediate product stream, and the substitute natural gas stream is formed from all of the mixed gas stream.

4. The method of claim 2, wherein:
a supplemental process stream is combined with the refinery gas stream to form the process stream; and
the supplemental process stream is nitrogen, air, hydrogen, natural gas or combinations thereof.

5. The method of claim 2, wherein:
the reactant stream is formed by combining the part of the process stream with part of a supplemental process stream;
the by-pass stream is formed by combining the remaining part of the process stream with the remaining part of the supplemental process stream; and
the supplemental process stream is nitrogen, air, hydrogen, natural gas or combinations thereof.

6. The method of claim 2, wherein the reactant stream is combined with steam to form the reactor feed stream.

7. The method of claim 2, wherein:
the by-pass stream is combined with the part of intermediate product stream;
the substitute natural gas stream is formed from the mixed gas stream;
a recycle stream is formed from a remaining part of the intermediate product stream either before all of the intermediate product stream is cooled or the part of the intermediate product stream is cooled; and
the recycle stream is introduced into the catalytic reactor.

8. The method of claim 2, wherein:
the by-pass stream is combined with all of the intermediate product stream after having been cooled;
the substitute natural gas stream is formed from the part of the mixed gas stream;
a recycle stream is formed from a remaining part of the mixed gas stream;
the recycle stream is combined with the reactant stream to form, at least in part, the reactor feed stream.

9. The method of claim 7 or claim 8, wherein steam is also combined with the reactant stream to form the reactor feed stream.

10. The method of claim 1, wherein the substitute natural gas stream is formed by combining at least part of the intermediate product stream with a supplemental process stream after the intermediate product stream is cooled and the supplemental process stream is nitrogen, air, hydrogen, natural gas or combinations thereof.

11. The method of claim 1, wherein one of the reactant stream and the reactor feed stream is also formed from a supplemental process stream and the supplemental process stream is nitrogen, air, hydrogen, natural gas, steam or combinations thereof.

12. An apparatus for producing a substitute natural gas stream to be added to a natural gas stream, said apparatus comprising:
a catalytic reactor configured to catalytically react hydrogen and the olefins within a reactor feed stream to produce an intermediate product stream containing saturated hydrocarbons formed from hydrogenation of the olefins;
a flow network connected to the catalytic reactor and having an inlet to receive the refinery gas stream, an outlet to discharge the substitute natural gas stream, a preheater positioned between the inlet and the catalytic reactor and an after-cooler positioned between the catalytic reactor and the outlet;
the flow network configured such that a reactant stream, formed at least in part from at least part of the refinery gas stream, is introduced into the preheater, the reactor feed stream is formed, at least in part, from the reactant stream after having been heated and the substitute natural gas stream is formed at least in part from at least part of the intermediate product stream after having been cooled in the after-cooler;
the preheater configured to heat the reactant stream such that the reactor feed stream has a temperature within a range of between 120° C. and 427° C. upon introduction into the catalytic reactor and the intermediate product stream has a discharge temperature upon discharge from the catalytic reactor within a range of between 204° C. and 650° C. and means for controlling an extent to which the hydrogen and olefins contained in the refinery gas stream are reacted in the catalytic reactor such that the substitute natural gas stream has a concentration of the olefins that is lower than that of the refinery gas stream and a greater interchangeability with the natural gas than the refinery gas.

13. The apparatus of claim 12, wherein the controlling means in part comprise:
a by-pass line having one end positioned within the flow network between the inlet and the preheater and the other end positioned between the after-cooler and the outlet and a flow control valve located within the by-pass line such that when the flow control valve is set in an open position the reactant stream is composed of part of a process stream that is composed at least in part of the refinery gas stream, a by-pass stream formed from at least a remaining part of the process stream flows within the by-pass line and combines with the at least part of the intermediate product stream to form a mixed gas stream and the substitute natural gas stream is formed, at least in part, from at least part of the mixed gas stream; and
the flow control valve configured to be progressively opened from a closed position such that flow of the by-pass stream increases and the olefins available for reaction within the catalytic reactor decreases.

14. The apparatus of claim 13, wherein the flow network is configured such that the process stream is composed solely of the refinery gas stream, the reactant stream is composed solely of the part of the process stream, the reactor feed stream is composed solely of the reactant stream, the by-pass stream is composed solely of the remaining part of the process stream, the by-pass stream is combined with all of the intermediate product stream, and the substitute natural gas stream is formed from all of the mixed gas stream.

15. The apparatus of claim 13, wherein:
the flow network has a supplemental inlet that is positioned between the inlet and the preheater to receive a supplemental process stream that combines with the refinery gas stream to form the process stream; and
the supplemental process stream is nitrogen, air, hydrogen, natural gas or combinations thereof.

16. The apparatus of claim 13, wherein:
the flow network has a supplemental inlet to receive a supplemental process stream, a first flow path and a second flow path;
the supplemental process stream is nitrogen, air, hydrogen, natural gas or combinations thereof;
the first flow path communicates, at one end with the supplemental inlet and at the other end to a position within the flow network between the inlet and the preheater such that the part of the process stream combines with part of the supplemental process stream to form the reactant stream; and
the second flow path communicates, at one end with the supplemental inlet and at the other end with the by-pass line, downstream of the flow control valve, such that the remaining part of the process stream combines with the remaining part of the supplemental process stream to form the by-pass stream.

17. The apparatus of claim 13, wherein the flow network has a supplemental inlet to receive steam, the supplemental inlet positioned between the inlet and the catalytic reactor such that the reactant stream combines with the steam to form the reactor feed stream.

18. The apparatus of claim 13, wherein:
a recycle line is positioned, at one end, between the catalytic reactor and the other end of the by-pass line and, at the other end, between the one end of the by-pass line and the catalytic reactor;
a blower positioned within the recycle line such that the remaining part of the intermediate product stream flows within the recycle line as a recycle stream back to the catalytic reactor;
the mixed gas stream is formed from the by-pass stream and the part of the intermediate product stream; and
the substitute natural gas stream is formed from the mixed gas stream.

19. The apparatus of claim 12, wherein:
the other end of the by-pass line is posited between the outlet and the after-cooler such that the by-pass stream combines with all of the intermediate product stream after having been cooled;
a recycle line is positioned, at one end, between the by-pass line and the outlet;
a blower is positioned within the recycle line such that the substitute natural gas stream is formed from the part of the mixed gas stream and a remaining part of the mixed gas stream flows into the recycle line as a recycle stream; and
the other end of the recycle line positioned between the one end of the by-pass line and the catalytic reactor such that the recycle stream flows back to the catalytic reactor.

20. The apparatus of claim 18 or claim 19 wherein the flow network has a supplemental inlet to receive steam, the supplemental inlet in communication with the recycle line such that the steam is also introduced into the catalytic reactor alone with the recycle stream.
